SEQUENCE LISTING (2 sequences)

SEQ ID NO: 1 - protein, 22 aa, Akkermansia muciniphila
  source 1..22: note = Novel strain of Akkermansia muciniphila deposited at the American Type Culture Collection (ATCC) under ATCC Accession No. PTA-126838.
MFLPCGGCRRAFLYPPGLFSGV

SEQ ID NO: 2 - protein, 65 aa, Akkermansia muciniphila
  source 1..65: note = Novel strain of Akkermansia muciniphila deposited at the American Type Culture Collection (ATCC) under ATCC Accession No. PTA-126838.
MNQLLSLESLRAYMIILQGEHHPKNKVAPARQTVTARNPRTMISFRYNTENNTVDAAKTGRDDTN

(19) United States
(12) Patent Application Publication
(10) Pub. No.: US 2025/0195586 A1
(43) Pub. Date: Jun. 19, 2025

(54) Novel Microbial Composition and Methods of Use Thereof

(71) Applicant: Pendulum Therapeutics, Inc., San Francisco, CA (US)

(72) Inventors: John S. Eid, San Francisco,, CA (US); Andrew T. Cheng, San Francisco, CA (US); Nicholas B. Justice, San Francisco, CA (US); Julia N. Myers, San Francisco, CA (US)

(21) Appl. No.: 18/847,148

(22) PCT Filed: Mar. 15, 2023
(86) PCT No.: PCT/US2023/064462

§ 371 Date: Sep. 13, 2024

Related U.S. Application Data
(60) Provisional application No. 63/320,000, filed on Mar. 15, 2022.

(51) Int. Cl. A61K 35/741; A61P 3/00; A61P 3/04; A61P 3/10; C12N 1/20; C12R 1/01

(52) U.S. Cl. CPC A61K 35/741; A61P 3/00; A61P 3/04; A61P 3/10; C12N 1/205; C12R 2001/01

(57) ABSTRACT

Provided herein is a novel Akkermansia muciniphila strain. Compositions comprising the strain are also provided. Methods comprising administering the compositions to a subject in need thereof are also provided. In some embodiments, provided are methods of treating a metabolic disorder in a subject, such methods comprising administering to the subject a composition of the present disclosure in an amount effective to treat the metabolic disorder in the subject. Methods comprising culturing the novel strain are also provided.

### CROSS-REFERENCE TO RELATED APPLICATIONS

This application is a national phase entry under 35 U.S.C. § 371 International Patent Application PCT/US2023/064462 filed Mar. 15, 2023, designating the United States of America and published in English as International Patent Publication WO 2023/178194 on Sep. 21, 2023, which claims the benefit of U.S. Provisional Patent Application No. 63/320,000, filed Mar. 15, 2022, which are incorporated herein by reference in their entirety.

### INCORPORATION BY REFERENCE

The ST.26 XML Sequence listing named “ST26 Sequence Listing.xml”, created on Mar. 15, 2023, and having a size of 4,096 bytes, is hereby incorporated herein by this reference in its entirety.

### BACKGROUND OF THE INVENTION

Akkermansia is a genus of gram-negative, strictly anaerobic, non-motile, non-spore-forming, oval-shaped bacterium. Culturing and formulating compositions comprising Akkermansia can be difficult due to the bacteria's strict anaerobic demands. Further, traditionally, Akkermansia has been cultured under anaerobic conditions on medium containing gastric mucin.

Provided herein is a novel strain of Akkermansia that grows without mucin in a vegetable media under anaerobic conditions.

### SUMMARY

The present disclosure provides bacteria of an Akkermansia muciniphila strain as deposited at the American Type Culture Collection (ATCC) under ATCC Accession No. PTA-126838. The bacteria comprise a nucleic acid encoding a polypeptide comprising the amino acid sequence set forth in SEQ ID NO:1, SEQ ID NO:2, or both.

The present disclosure provides a composition comprising bacteria of an Akkermansia muciniphila strain as deposited at the American Type Culture Collection (ATCC) under ATCC Accession No. PTA-126838. The composition, as presented herein, comprises a nucleic acid encoding a polypeptide comprising the amino acid sequence set forth in SEQ ID NO:1, SEQ ID NO:2, or both.

Also presented herein is a composition for use in treating a metabolic disorder in a subject, wherein the composition comprises the bacteria of an Akkermansia muciniphila strain as deposited at the American Type Culture Collection (ATCC) under ATCC Accession No. PTA-126838 in an effective amount to treat the metabolic disorder. In an embodiment, the metabolic disorder is selected from metabolic disorder is selected from the group consisting of or consisting essentially of: insulin-resistance based disorders, insulin-sensitivity based disorders, type-1-diabetes, type-2-diabetes, and obesity.

In certain embodiments, the composition further comprises one or more additional microbes having a 16S rRNA sequence comprising at least 97% identity to the full length of a 16S rRNA sequence of a microbe selected from the group consisting of: Anaerostipes caccae, Anaerobutyricum hallii, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and any combinations thereof.

In certain embodiments, the composition further comprises one or more additional microbes selected from the group consisting of: Anaerobutyricum hallii, Clostridium butyricum, Clostridium beijerinckii, Clostridium butyricum, and any combination thereof.

In certain embodiments, the composition comprises at least 10\hat{ } 5 AFUs/g of each of the one or more additional microbes.

In further embodiments, the composition comprises at least one preservative and/or an enteric coating. In some embodiments, the composition comprises a prebiotic chosen from the group consisting of inulin, green banana, reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, b-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof. In some preferred embodiments, the prebiotic used is inulin.

In some embodiments, presented herein are compositions comprising bacteria that are lyophilized. In some embodiments, compositions presented herein, comprise a bacteria of an Akkermansia muciniphila strain as deposited at the American Type Culture Collection (ATCC) under ATCC Accession No. PTA-126838 that is lyophilized.

In some embodiments, compositions presented herein comprise bacteria that are viable. In some embodiments, compositions presented herein, comprise a bacteria of an Akkermansia muciniphila strain as deposited at the American Type Culture Collection (ATCC) under ATCC Accession No. PTA-126838 that is viable.

In some embodiments, compositions presented herein comprise bacteria that are non-viable. In some embodiments, compositions presented herein, comprise a bacteria of an Akkermansia muciniphila strain as deposited at the American Type Culture Collection (ATCC) under ATCC Accession No. PTA-126838 that is non-viable.

In some embodiments, the composition is formulated as a pharmaceutical formulation. In some embodiments, the composition is formulated as a nutritional supplement. In some embodiments, the composition is formulated as a dietary supplement. In some embodiments, the composition is formulated as a medical food.

In some embodiments, presented herein, is a composition that is dairy-free. In some embodiments, the composition comprises no animal products.

In some embodiments, the composition is in the form of a pill, a capsule, a lozenge, a food bar, or a gummy ball.

Provided herein are also methods of treating a metabolic disorder in a subject, the method comprising administering to the subject the compositions comprising Akkermansia muciniphila strain as deposited at the American Type Culture Collection (ATCC) under ATCC Accession No. PTA-126838. The composition can further comprise of Anaerobutyricum hallii, Clostridium butyricum, Clostridium beijerinckii, Clostridium butyricum, and any combination thereof.

The metabolic disorders referred to in the methods of treatment selected from insulin-resistance based disorders, insulin-sensitivity based disorders, type-1-diabetes, type-2-diabetes, and obesity, gut-related disorders.

Provided herein are methods of culturing Akkermansia muciniphila strain as deposited at the American Type Culture Collection (ATCC) under ATCC Accession No. PTA-126838 under anaerobic growth conditions in/on a growth medium. The growth medium used herein is a VEG-medium.

### BRIEF DESCRIPTION OF THE DRAWINGS

FIG. 1: Depicts the comparison of growth phase between Akkermansia muciniphila strains Amuc-OG and Amuc-i09.

FIG. 2: Depicts growth curves for three independent replicate comparisons each betweenA. muciniphila (OG) and Amuc-i09.

FIG. 3: Depicts plot for doubling time, pH consumption, and glucose consumption comparisons between Amuc-i09 and Amuc-OG during growth phase.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise.

### Strain Deposit

The following biological material has been deposited under the terms of the Budapest Treaty with the American Type Culture Collection (ATCC®), Patent Depository
P0071744 at 10801 University Boulevard Manassas, Virginia 20110, and given the following accession number: PTA-126838.

| Name/Designation | Patent Deposit Number | Patent Deposit No. |
|---|---|---|
| Akkermansia muciniphila | WB-STR-i09 | PTA- 126838 |

### A. BACTERIA

Described herein are new strains of anaerobic bacteria. In some embodiments, bacteria as described herein is the strain as deposited at the ATCC®, under ATCC® deposit number PTA-126838 (herein referred to as Amuc-i09).

Provided herein is a novel strain of Akkermansia muciniphila. The strain as disclosed herein is as deposited at the American Type Culture Collection (ATCC) under ATCC Accession No. PTA-126838. The genome of the bacteria of the Akkermansia muciniphila strain as deposited at the ATCC® under ATCC® Accession No. PTA-126838 encodes a polypeptide comprising SEQ ID NO: 1 and a polypeptide comprising SEQ ID NO: 2:

Sequence Information
SEQ ID NO: 1
MFLPCGGCRRAFLYPPGLFSGV
SEQ ID NO: 2
MNQLLSLESLRAYMIILQGEHHPKNKVAPARQTVTARNPRTMISFRYNTE
NNTVDAAKTGRDDTN

### B. ADDITIONAL BACTERIA

Embodiments of compositions described herein include but are not limited to, compositions comprising Amuc-i09 alone or in combination with one more other bacteria. Examples of bacteria which may be combined in a composition with Amuc-i09 include, but are not limited to: Clostridium butyricum, Clostridium beijerinckii, Anaerostipes caccae, Anaerobutyricum hallii, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combinations thereof.

In certain embodiments, compositions comprising Amuc-i09 further comprise bacteria of a genus selected from the group consisting of Clostridia, Eubacteria, Bifidobacteria, Anaerostipes, Coprococcus, Bacteroides, Blautia, Ruminococcus, Faecalibacterium, Oscillospira, Streptococcus, and Roseburia.

In one embodiment, a composition comprises of Amuc-i09 and one or more additional microbes selected from the group consisting of Anerobutyricum hallii DSM 3353, Akkermansia muciniphila ATCC BAA83, Clostridium butyricum ATCC 1939, Clostridium beijerinckii ATCC 5174, and any combinations thereof.

In one embodiment, a composition comprises of Amuc-i09 and one or more additional bacteria strains selected from the group consisting of Anerobutyricum hallii, Akkermansia muciniphila, Clostridium butyricum, Clostridium beijerinckii, and any combinations thereof.

In one embodiment, the additional bacteria strains in compositions therein are anaerobic bacteria. In another embodiment, the additional bacteria are not viable. In still other embodiments, the additional bacteria are lyophilized. In other embodiments, the additional bacteria are lyophilized and viable.

In another embodiment, the present disclosure provides compositions comprising Amuc-i09, and at least one additional microbe with a 16S rRNA sequence comprising at least about an 85%, 87%, 90%, 92%, 94%, 95%, 96%, 97%, 98%, 99%, or 100% sequence identity to the full length of a 16S rRNA sequence of a microbe selected from the group consisting of: Clostridium butyricum, Clostridium beijerinckii, Anaerostipes caccae, Anaerobutyricum hallii, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combinations thereof.

In another embodiment, described herein is bacteria that express a polypeptide comprising the amino acid sequence set forth in SEQ ID NO:1, a polypeptide comprising the amino acid sequence set forth in SEQ ID NO:2, or both. In some embodiments, such bacteria is an Akkermansia muciniphila strain of bacteria.

### C. PROPERTIES OF BACTERIA

In embodiments, the novel strain of Akkermansia muciniphila as disclosed herein, Amuc-i09, shows unique phenotypic properties. Such phenotypic properties include improved viability (measured in CFUs per gram), faster and/or improved growth characteristics of Amuc-i09 compared to other Akkermansia strains, like Amuc-OG.

Properties of growth of frozen or lyophilized strains are measured and compared to a base strain. Such a base strain may be one that is deposited with a certified depositing institution or any other starting material. In a particular embodiment, growth properties of lyophilized strain(s) of Amuc-i09 are measured and compared to the properties of Amuc-OG.

a. Viability

Viability, as defined herein, is the measure of live cells. Specifically, viability is defined as the ability to multiply via binary fission, under controlled conditions. Viability is measured in Colony Forming Units (CFUs) and is measured by culturing the microbes and counting only viable cells. Another unit used is Active Fluorescence Units (AFUs) that measure viability based on flow cytometer results that correlate cell wall integrity to live cells.

Viability may be measured using flow cytometry (BD Accuri™ C6). Such flow cytometry measurements include staining the cell mixture and detecting the fluorescence of each cell as it flows by. The staining of the cell mixture can be done using any of the stains that preferentially stain cells, especially live and dead cells, or just dead cells. Examples of such stains include, but are not limited to, Thiazole Orange, Propidium Iodide, 7-AAD, DAPI, SYTO 9, fixable dyes like Zombie Aqua™, Zombie Green™ Zombie NIR™, Zombie Red™, Zombie Violet™, Zombie UV™, and Zombie Yellow™ or any combinations thereof. In one embodiment, the stain used herein for processes of this disclosure is a combination of thiazole orange and Propidium Iodide.

As disclosed herein, Amuc-i09 displays a greater viability per gram compared to the wild type variant, Amuc-OG. For instance, as shown in the examples, 4 g of dry weight of Amuc-i09 has 8.72×10\hat{ }11 CFUs while only 8.29×10\hat{ }11 CFUs of Amuc-OG is present in 21.3 g of dry weight of Amuc-OG. Calculated per gram, Amuc-i09 has 2.18×10\hat{ }11 Cfus/g of the microbe, while Amuc-OG has only 0.38×10\hat{ }11 CFUs/g of the Amuc-OG microbe. This also signifies that per gram of the Amuc-i09 has more live bacteria than per gram of the Amuc-OG strain.

In some embodiments, the Amuc-i09 are in a viable, lyophilized state. In some embodiments, the bacteria of the present disclosure are not viable.

In further embodiments, one or more of the bacteria in any of the compositions described herein are in a lyophilized state. Further, one or more of the bacteria in any of the compositions described herein are viable or non-viable.

In some embodiments, the bacteria of the present disclosure are present at a concentration of approximately 1×10³ to 1×10¹⁴ Active Fluorescence Units (AFU)/g, inclusive of any numbers within the range, whereas in other embodiments the concentrations are approximately 1×10⁹ to 1×10¹³ AFU/g, approximately 1×10⁵ to 1×10⁷ AFU/g, or approximately 1×10⁸ to 1×10⁹ AFU/g, inclusive of any numbers within the range.

In certain embodiments, compositions of the present disclosure comprise at least 10\hat{ }5 CFUs of each strain per gram. In a further embodiment, compositions comprise at least 10\hat{ }6 CFUs/g, or at least 10\hat{ }7 CFUs/g, or at least 10\hat{ }8 CFUs/g, or at least 10\hat{ }9 CFUs/g, or at least 10\hat{ }10 CFUs/g, or at least 10\hat{ }11 CFUs/g, or at least 10\hat{ }12 CFUs/g of at least one strain in the composition. In other embodiments, wherein there are more than one strain, each strain may have a viability (measured in CFUs or AFUs) different from the other strains in the composition.

b. Faster and/or Improved Growth Characteristics

Amuc-i09, as disclosed herein, has faster growth properties compared to the wild-type strain. Growth is measured in terms of OD or optical density of the media in which the microbe is cultured.

The term “faster growth”, as used herein, is wherein the doubling time of a particular organism is smaller than another organism under otherwise identical conditions.

In some embodiments, Amuc-i09 has improved growth characteristics compared to the wild type strain, Amuc-OG. The term “improved”, as used herein, refers to a quantitative improvement in the number of live cells after a certain time point in the growth phase of the bacteria. Specifically, Amuc-i09 is shown herein to have more number of live cells (measured in CFU/g) after a time point of, for e.g., 9 h or 12 h compared to the number of live cells (measured in CFU/g) of Amuc-OG after the same time, under conditions wherein both the bacteria are grown in same conditions.

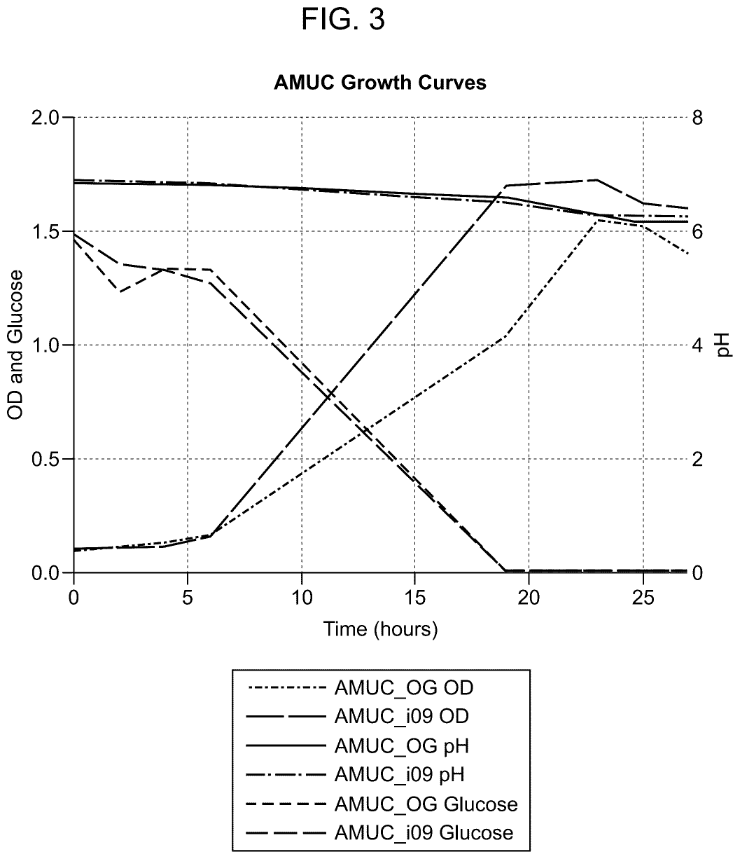

As is described in the example 1 and FIG. 3, Amuc-i09 is characterized by a faster doubling time as compared to Amuc-OG at the exponential growth phase.

c. Improved Pelleting Properties

In some embodiments, Amuc-i09, as disclosed herein, has improved pelleting properties compared to the pelleting properties of Amuc-OG.

“Pelleting properties” as used herein, refers to the percentage of organisms in a liquid culture that are present in a recoverable cell pellet after centrifugation. An organism with “improved pelleting properties”, when centrifuged, would have a greater ratio of the organism present in the pellet versus the supernatant (pellet:supernatant) as compared to the pellet:supernatant ratio of another organism pelleted under otherwise identical conditions. In embodiments presented herein, Amuc-i09 is shown to have a greater pellet:supernatant ratio compared to Amuc-OG, when cultured and pelleted under otherwise identical conditions.

In certain embodiments, Amuc-i09 exhibits “improved pelleting properties” by exhibiting a supernatant that is more (visually) transparent compared to the supernatant of the Amuc-OG organism pelleted under otherwise identical conditions.

Specifically, as described in Example 2 and illustrated in Table 2, Amuc-i09 displayed improved pelleting properties as compared to Amuc-OG.

### D. METHODS OF USE

In certain embodiments, the present disclosure provides methods of treating a disorder. Such a disorder may be a gut-based disorder or any other disorder based on an imbalance of bacterial population in the gut. By way of non-limiting example, such a population may be an Akkermansia muciniphila population in the gut.

In certain embodiments, the present disclosure provides methods of increasing the population of Akkermansia muciniphila in the small intestine.

In further embodiments, the compositions disclosed herein may be used to treat any one or more of the disorders like irritable bowel syndrome, inflammatory bowel disease, a stomach ulcer, pouchitis, Helicobacter pylori infection, diarrhea, type-1-diabetes mellitus, type-2-diabetes mellitus, and/or obesity.

In embodiments, compositions described herein may include a pharmaceutically acceptable carrier suitable for oral administration to a mammal, for example, as a powdered food supplement, a variety of pelletized formulations, or a liquid formulation. In some embodiments, the composition is in the form of a pill, a capsule, a lozenge, a food bar, and/or a gummy ball. In some embodiments, the composition is in the form of a suppository, or an injection for delivery to a mammal.

In some embodiments, the compositions described herein may be used as a nutritional supplement or a dietary supplement for a mammal. In some instances, the mammal can be a human.

### E. METHODS OF MAKING

Embodiments include methods of generating bacteria that have improved growth characteristics and improved pelleting properties. Such bacteria may be obtained by a process that involves isolating the bacteria from a bio-sample, inoculating and culturing it under suitable anerobic conditions in a suitable media, pelleting with cryoprotectant, freezing, lyophilizing, and resuspending in media.

In some embodiments, bacteria are grown in any suitable medium; some non-limiting examples include PYG, RCM, GYT veg, BHI, nutrient media, minimal media, selective media, and differential media. In a particular embodiment, the medium is a vegetable-based medium. As used herein, a “vegetable-based medium” is a growth medium that is essentially free of any animal or dairy based ingredients or derivatives. In some embodiments, the vegetable-based medium is a meat-free medium that is free of any animal-derived component. In some embodiments, the vegetable-based medium is a liquid “VEG-medium” with components as shown in Table 1:

TABLE 1
Composition of VEG-medium

| Component | Amount per liter | |
|---|---|---|
| Sodium Chloride | 4.9 | g |
| Sodium Bicarbonate | 0.4 | g |
| Monopotassium phosphate | 0.04 | g |
| Magnesium Sulfate Heptahydrate | 0.02 | g |
| Potassium Phosphate, Dibasic | 2.04 | g |
| Calcium Chloride | 0.02 | g |
| Dextrose | 2 | g |
| Sodium Phosphate Dibasic | 2.5 | g |
| Cysteine-HCl | 0.5 | g |
| N-Acetylglucosamine | 2 | g |
| HiVeg Special Infusion | 7.5 | g |
| HiVeg Extract #2 | 10 | g |
| HiVeg Peptone #3 | 10 | g |
| AntiFoam | 0.05 | ml |
| D-Biotin | 0.0002 | g |
| Calcium Pantothenate (4C) | 0.0026 | g |
| Myoinositol | 0.02 | g |
| p-Aminobenzoic Acid | 0.0006 | g |
| Pyridoxine Hydrochloride | 0.005 | g |
| Riboflavine | 0.0006 | g |
| Thiamine Hydrochloride | 0.01 | g |
| Vitamin B12 (4C) | 0.0002 | g |
| Nicotinic acid | 0.005 | g |

Cryoprotectants useful in the present method include, but are not limited to, lactate, trehalose, glycerol, DMSO, propylene glycol, 2-methyl-2, 4-pentanediol, methanamide, glycerophospholipids, proline, sorbitol, Diethyl glycol, sucrose, glucose, and polymers like polyvinyl alcohol, PEG, hydroxyethyl starch, skim milk, tapioca, Polyvinyl-propiline, Inulin, Methylcellulose, Sodium Alginate, Gum Arabic, Propylene Glycol, or Xylitol and all possible combinations thereof. In embodiments, a cryoprotectant is added to the bacteria before freezing.

In particular embodiments, the bacterial pellet is resuspended in a cryoprotectant. In some embodiments, the cryoprotectant used is sucrose.

The cryoprotected bacteria was then resuspended in the VEG-medium and centrifuged to obtain pellets. In comparing the bacterial pellet between Amuc-OG and Amuc-i09, Amuc-i09 was surprisingly found to have improved pelleting properties compared to Amuc-OG.

Following resuspension, the cryo-suspended bacteria pellet is frozen. For example, the pellet may be flash-frozen in liquid nitrogen. Other freezing techniques that a person of ordinary skill in the art may employ here—including cryogenic (e.g., in ethanol and dry ice, or carbon dioxide), mechanical freezing, or flash-freezing, are contemplated as part of this disclosure.

In certain embodiments, the mixture of cells and cryoprotectant is lyophilized. Such lyophilization may occur over a period of 12-48 h. Lyophilization may be performed under a vacuum and at a temperature below freezing. A non-limiting example of lyophilization conditions is 0.008 mbar and a temperature of −84° C. Other lyophilization conditions that may be altered include the surface area of the product exposed to sublimation, controlling the pressure and temperature of the lyophilization chamber, and altering the amount of heat applied to the product both during sublimation throughout the primary drying phase and throughout desorption in the secondary drying phase.

In a particular embodiment, the bacteria pellet is lyophilized in a LabConco FreeZone 2.5Plus lyophilizer for 8-24 hr at −84° C. and a vacuum pressure of 0.008 to 0.1 mbar.

Lyophilized or frozen bacteria are then revived. In embodiments, the bacteria is revived by placing the bacteria in a culture media and incubating under growth conditions. In a particular embodiment, bacteria in a proportion of ˜0.01 mg/mL are revived by placing the lyophilized cells in vegetable media (as defined above) and allowing the culture to grow for 12-20 h at a temperature suitable for bacterial growth. This process selects for the cells that are capable of reviving rapidly from freezing or lyophilization. The process may then be repeated through additional cycles to further select a bacterial population.

At the end of each cycle, the bacteria are revived, frozen or lyophilized and grown in media under suitable conditions. A comparison study was conducted to identify growth characteristic differences between the novel Amuc-i09 strain and Amuc-OG.

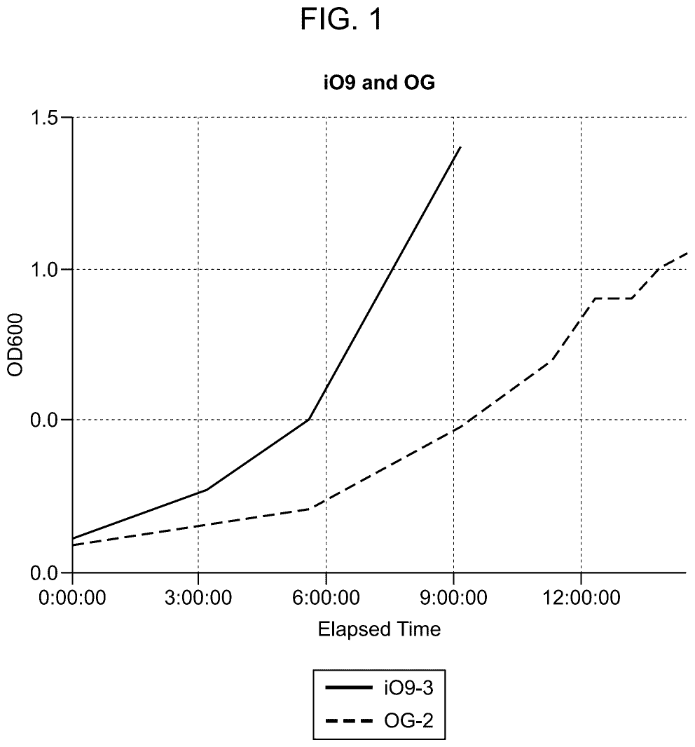

As described herein, the novel Amuc-i09 strain was found to have improved growth characteristics compared to Amuc-OG. The improved growth characteristics refers to a faster growth phase as characterized by FIG. 1 that compares the growth characteristics of Amuc-i09 (“i09-3” is a third replicate of Amuc-i09 grown in the media as described above) vs. Amuc-OG when grown in the same media as described above.

As is evident, in FIG. 1, the i09-3 (Amuc-i09 batch 3) grows to a much higher OD within 9 hrs while OG-2 (Amuc-OG batch 2) doesn't reach the same high OD values even after 12 hrs.

In certain embodiments, a single colony of the bacteria or a sample of bacteria is revived and grown in the vegetable media as described above. The number of cycles a selected colony may go through may be at least 5 cycles, at least 10 cycles, at least 15 cycles, at least 20 cycles, at least 25 cycles, at least 30 cycles, at least 35 cycles, at least 40 cycles, at least 45 cycles, at least 50 cycles, at least 55 cycles, at least 60 cycles, at least 65 cycles, at least 70 cycles, at least 75 cycles, at least 80 cycles, at least 85 cycles, at least 90 cycles, at least 95 cycles, at least 100 cycles, at least 105 cycles, at least 110 cycles, at least 115 cycles, at least 120 cycles, at least 125 cycles, at least 130 cycles, at least 135 cycles, at least 140 cycles, at least 145 cycles, or at least 150 cycles. Each cycle may present one or more challenges to the bacteria. Examples of such challenges include, but are not limited to, changes in growth conditions, nutrient medium ingredients, temperature, pressure, oxygen conditions to the process of isolation, freezing and/or lyophilization, and/or revival of microbes as described herein.

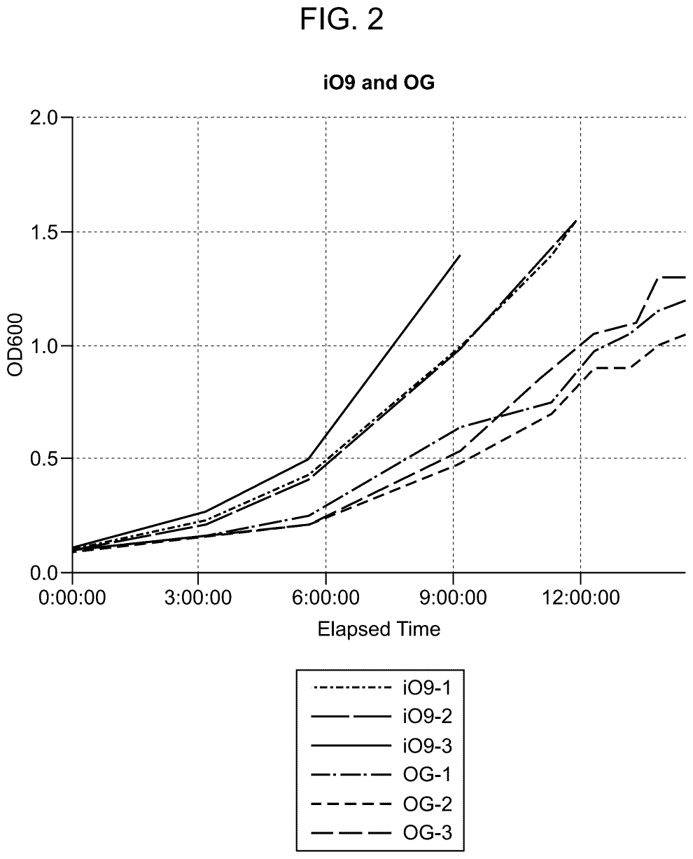

In one embodiment, lyophilized powder of each of the Amuc-i09 strain and Amuc-OG strain was revived on an agar plate. From a single colony of each of the strains, 3 replicates were cultured in the vegetable media as described herein. The data showing the growth phase of each of those replicates is as shown in FIG. 2.

### F. COMPOSITIONS

Provided herein are compositions that comprise one or more bacteria of the present disclosure. Such compositions may be administered to a subject. In certain embodiments, such compositions may be administered as therapeutics or as a dietary supplement. One or more bacteria of the present disclosure, as described herein, may be used to create a pharmaceutical formulation comprising an effective amount of a bacteria for treating a subject. The microorganisms may be in any formulation known in the art. Some non-limiting examples of such formulations include topical, capsule, pill, enema, liquid, injection formulations, and the like. In some embodiments, the one or more strains disclosed herein may be included in a food or beverage product, cosmetic, or nutritional supplement.

In some embodiments, the subject is a mammal. Mammal, as used herein, refers to any mammal, including but not limited to, human, mouse, cat, rat, dog, sheep, monkey, goat, rabbit, hamster, horse, cow or pig. In a preferred embodiment, the mammal is human.

The composition may include one or more active ingredients. Active ingredients include, but are not limited to, those selected from the group consisting of: antibiotics, prebiotics, probiotics, glymays (e.g., as decoys that would limit specific bacterial/viral binding to the intestinal wall), bacteriophages, microorganisms and the like.

In some embodiments, the composition comprises a prebiotic. The prebiotic may be as a combination or as a single ingredient. In some embodiments, the prebiotic may be a source of starch, mucin, fructo-oligosaccharides, pectin, gums, glucans, xylans, arabinogalactan, seaweed polysaccharides, or derivatives thereof. Some examples of such sources are inulin, green banana, reishi, tapioca, oats, pectin, raw or cooked potato, maize, rice, rice bran, cereals, porcine or human sources of mucin, derivatives of L-threonine, chicory root, agave, artichoke, dandelion, lemon peel, apple peel, berries, guar gum, xanthan gum, acacia chia, barley, sorghum, corn, larch, arabinex, kelp, dulse or any combinations or derivatives thereof. In some embodiments, the prebiotic is inulin. Inulin serves as an energy source for the microbial composition.

In some embodiments, the prebiotic is selected from the group consisting of inulin, green banana, reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, b-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof.

In some other embodiments, olive oil polyphenols including hydroxytyrosol and its derivatives, e.g., oleuropein complex and tyrosol (“phenolic alcohols”) are used as a prebiotic.

A composition may be administered by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract.

In some embodiments, administration of a composition occurs orally, for example, through a capsule, pill, powder, tablet, gel, or liquid, designed to release the composition in the gastrointestinal tract. In some embodiments, administration of a composition occurs by injection, for example, for a composition comprising butyrate, propionate, acetate, and short chain fatty acids. In some embodiments, the administration of a composition occurs by application to the skin, for example, cream, liquid, or patch. In some embodiments, administration of a composition occurs by a suppository and/or by enema. In some embodiments, a combination of administration routes is utilized.

Microbial compositions may be formulated as a dietary supplement. Microbial compositions may be incorporated with vitamin supplements. Microbial compositions may be formulated in a chewable form such as a probiotic gummy. Microbial compositions may be incorporated into a form of food and/or drink. Non-limiting examples of food and drinks where the microbial compositions may be incorporated include, for example, bars, shakes, juices, infant formula, beverages, frozen food products, fermented food products, and cultured dairy products such as yogurt, yogurt drink, cheese, acidophilus drinks, and kefir.

A composition of the disclosure may be administered as part of a fecal transplant process. A composition may be administered to a subject by a tube, for example, nasogastric tube, nasojejunal tube, nasoduodenal tube, oral gastric tube, oral jejunal tube, or oral duodenal tube. A composition may be administered to a subject by colonoscopy, endoscopy, sigmoidoscopy, and/or enema.

In some embodiments, the microbial composition is formulated such that the one or more microbes may replicate once they are delivered to the target habitat (e.g. the gut). In one non-limiting example, the microbial composition is formulated in a pill. Such a pill may have a shelf life of at least 1, 2, 3, 4, 5, 6, 7, 8, 9, 10, 11, or 12 months. In another non-limiting example, the microbial composition is formulated so that the microbes may reproduce once they are in the gut.

In some embodiments, the Amuc-i09 may be formulated with other additives in a pill that may be administered orally to an individual, such that the pill does not disintegrate until the pill reaches the intestine of the individual. In some embodiments, other components may be added to aid the shelf life of the microbial composition. In some embodiments, Amuc-i09 may be formulated in a manner that it is able to survive in a non-natural environment. For example, a microbe that is native to the gut may not survive in an oxygen rich environment. To overcome this limitation, the microbe may be formulated to reduce or eliminate the exposure to oxygen. Other strategies to enhance the shelf-life of microbes may include other microbes (e.g., whereby one or more strains in the composition improves the survival of one or more of the other strains in the composition).

In some embodiments, Amuc-i09 may be formulated with polyphenols. In some embodiments, the polyphenols may be a combination of one or more polyphenols. Commonly used polyphenols are flavonoids that include flavones, flavonols, flavanols, flavanones, isoflavones, proanthocyanidins, and anthocyanins. Particularly abundant flavanoids in foods are catechin (tea, fruits), hesperetin (citrus fruits), cyanidin (red fruits and berries), daidzein (soybean), proanthocyanidins (apple, grape, cocoa), and quercetin (onion, tea, apples). Phenolic acid include caffeic acid, while lignans are polyphenols derived from phenylalanine found in Flax seed and other cereals. Tannins obtained from papaya is used in one embodiment.

In some embodiments, Amuc-i09 is lyophilized.

In some embodiments, Amuc-i09 is lyophilized and formulated as a composition, as provided herein. Such a composition may be formulated as a powder, tablet, enteric-coated capsule (e.g., for delivery to ileum/colon), or pill that may be administered to a subject by any suitable route. In some embodiments, the lyophilized formulation may be mixed with a saline or other solution prior to administration.

In some embodiments, wherein the composition comprises of Amuc-i09, wherein the bacteria is completely viable (comprising 100% live population of cells), is partially viable (comprising <50% of live population of cells) or is mostly non-viable (comprising >50% of dead population of cells).

In some embodiments, a composition is provided that comprises Amuc-i09 and Amuc-OG. In some embodiments, the composition comprises at least 50% of live Amuc-i09 bacteria. In some embodiments, the composition comprises at least 50% of dead Amuc-i09. In some embodiments, the composition comprises of Amuc-i09-derived extracellular vesicles.

In some embodiments, a microbial composition is formulated for oral administration, for example, as an enteric-coated capsule or pill, for delivery of the contents of the formulation to the ileum and/or colon regions of a subject.

### I. Capsule Ingredients

In some embodiments, the microbial composition is formulated for oral administration. In some embodiments, the microbial composition is formulated as an enteric coated pill or capsule for oral administration. In some embodiments, the microbial composition is formulated for delivery of the microbes to the ileum region of a subject. In some embodiments, the microbial composition is formulated for delivery of the microbes to a colon region (e.g., upper and/or lower colon) of a subject. In some embodiments, the microbial composition is formulated for delivery of the microbes to the ileum and colon regions of a subject.

In an embodiment, a composition comprising Amuc-i09 is administered as a capsule for oral delivery.

In an embodiment, a composition comprising a combination of Amuc-i09 and Amuc-OG is administered as a capsule for oral delivery.

In an embodiment, a composition comprising a combination of Amuc-i09 and another anaerobic bacteria is administered as an oral formulation.

In an embodiment, the size of the capsule is size 0, although the capsule size may be any size that is suitable for administration to a subject. Further, in the embodiment, the capsule has an enteric coating. An enteric coating may protect the contents of the oral formulation from the acidity of the stomach and provide delivery to the ileum and/or upper colon regions. Non-limiting examples of enteric coatings include pH sensitive polymers (e.g., eudragit FS30D), methyl acrylate-methacrylic acid copolymers, cellulose acetate succinate, hydroxypropyl methyl-cellulose phthalate, hydroxypropyl methyl-cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers. In some embodiments, the enteric coating is formed by a pH sensitive polymer. In some embodiments, the enteric coating is formed by eudragit FS30D.

The enteric coating may be designed to dissolve at any suitable pH. In some embodiments, the enteric coating is designed to dissolve at a pH greater than about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating may be designed to dissolve at a pH greater than: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

In some embodiments, the administration of a formulation of the disclosure may be preceded by, for example, colon cleansing methods such as colon irrigation/hydrotherapy, enema, administration of laxatives, dietary supplements, dietary fiber, enzymes, and magnesium or derivatives thereof.

Formulations provided herein may include the addition of one or more agents to the therapeutics or cosmetics in order to enhance stability and/or survival of the microbial formulation. Non-limiting example of stabilizing agents include genetic elements, glycerin, ascorbic acid, skim milk, lactose, tween, alginate, xanthan gum, carrageenan gum, mannitol, palm oil, and poly-L-lysine (POPL).

### II. Microbial Formulations and Compositions in Microbial Consortia

Compositions as disclosed here may comprise of a single microbial species or a combination of microbes. In one embodiment, the formulation comprises of only Amuc-i09. In a further embodiment, the formulation comprises of Amuc-i09 and at least one microbe chosen from Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus inf antis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and any combination thereof.

In another embodiment, the composition as disclosed herein, comprises of one or more recombinant microbes or microbes that have been genetically modified. In other embodiments, one or more microbes are not modified or recombinant. In some embodiments, the composition comprises microbes that may be regulated, for example, a microbe comprising an operon or promoter to control microbial growth. Microbes, as disclosed herein, may be produced, grown, or modified using any suitable methods, including recombinant methods.

In an embodiment, the population of the bacteria that may be administered as a part of the compositions as disclosed here, may comprise of any one or more of the species selected from the group consisting of: Amuc-i09, Amuc-OG, any other strain of Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus inf antis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and any combination thereof.

In some embodiments, provided are therapeutic compositions to treat a metabolic disorder comprising a purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Amuc-i09, Eubacterium hallii DSM 3353, Akkermansia muciniphila ATCC BAA83, Clostridium butyricum ATCC 1939, Clostridium beijerinckii ATCC 5174, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combination thereof.

In some embodiments, the metabolic disorder may be selected from insulin-resistance based disorders, insulin-sensitivity based disorders, type-1-diabetes, type-2-diabetes, and obesity.

In some embodiments, provided are therapeutic compositions to treat type-2-diabetes or obesity comprising a purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Amuc-i09, Eubacterium hallii DSM 3353, Akkermansia muciniphila ATCC BAA83, Clostridium butyricum ATCC 1939, Clostridium beijerinckii ATCC 5174, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combination thereof.

In some embodiments, provided are therapeutic compositions to treat type-2-diabetes or obesity comprising a purified microorganism population comprising a bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of Amuc-i09.

In some embodiments, provided are therapeutic compositions to treat type-2-diabetes or obesity comprising a purified microorganism population comprising a bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of Amuc-OG.

In some embodiments, provided are therapeutic compositions to treat a neurological or behavioral disorder comprising a purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: Amuc-i09, Eubacterium hallii DSM 3353, Akkermansia muciniphila ATCC BAA83, Clostridium butyricum ATCC 1939, Clostridium beijerinckii ATCC 5174, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combination thereof.

In some embodiments, the neurological or behavioral disorder is selected from gut-brain axis disorders, anxiety, or hyperalgesia. In some embodiments, the composition disclosed herein modulates neurotransmitter levels in the brain of a subject.

In some embodiments, provided are therapeutic compositions to treat a neurological or behavioral disorder comprising a purified microorganism population consisting of Amuc-i09.

In some embodiments, provided are therapeutic compositions to treat a gut-related disorder comprising a purified microorganism population consisting of Amuc-i09. In some embodiments, the gut-related disorder is selected from constipation, bloating, gut-injury related disorders, diarrhea, gastritis, inflammation of the gut, Crohn's disease, irritable bowel syndrome, inflammatory bowel disease, or dysbiosis.

In some embodiments, a therapeutic consortium comprises Akkermansia muciniphila, Bifidobacterium adolescentis, Bifidobacterium infantis, Clostridium beijerinckii, Clostridium butyricum, and Amuc-i09.

In some embodiments, a therapeutic consortium comprises Akkermansia muciniphila and Eubacterium hallii or Anerobutyricum hallii.

In some embodiments, a therapeutic consortium comprises Amuc-i09 and Eubacterium hallii or Anerobutyricum hallii.

In some embodiments, a therapeutic consortium comprises Amuc-i09 and any one of Bifidobacterium adolescentis, Bifidobacterium infantis, Clostridium beijerinckii, and Clostridium butyricum.

A therapeutic composition may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, or at least 50, or at least 75, or at least 100 types of bacteria. A therapeutic composition may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 45, or at most 50, or at most 75, or at most 100 types of bacteria.

In some embodiments, combining one or more microbes in a therapeutic composition or consortia may provide a synergistic effect when administered to the individual.

For example, administration of a first microbe may be beneficial to a subject and administration of a second microbe may be beneficial to a subject but when the two microbes are administered together to a subject, the benefit is greater than either benefit alone.

Different types of microbes in a therapeutic composition may be present in the same amount or in different amounts. For example, the ratio of two bacteria in a therapeutic composition may be about 1:1, 1:2, 1:5, 1:10, 1:25, 1:50, 1:100, 1:1000, 1:10,000, or 1:100,000.

### III. Compositions Comprising Probiotics

In an embodiment, a composition comprising one single microbe as a probiotic is disclosed. In another embodiment, a composition comprising consortia of bacteria, comprising at least two different bacteria is disclosed. Apart from the probiotic, the composition, may comprise a prebiotic, an antibiotic, or a combination of active agents as described herein.

In some embodiments, a composition is administered before, during, and/or after treatment with an antimicrobial agent such as an antibiotic. For example, the composition may be administered at least about 1 hour, 2 hours, 5 hours, 12 hours, 1 day, 3 days, 1 week, 2 weeks, 1 month, 6 months, or 1 year before and/or after treatment with an antibiotic. The formulation may be administered at most 1 hour, 2 hours, 5 hours, 12 hours, 1 day, 3 days, 1 week, 2 weeks, 1 month, 6 months, or 1 year before and/or after treatment with an antibiotic.

In some embodiments, the composition as disclosed herein, is administered during, after, and/or before with an anti-diabetic agent like insulin or a drug like metformin. For example, the formulation may be administered at least about 1 hour, 2 hours, 5 hours, 12 hours, 1 day, 3 days, 1 week, 2 weeks, 1 month, 6 months, or 1 year before and/or after treatment with the anti-diabetic agent.

In some embodiments, a composition is administered before, during, and/or after food intake by a subject. In some embodiments, the composition is administered with food intake by the subject. In some embodiments, the composition is administered with (e.g., simultaneously) with food intake.

In some embodiments, the composition is administered before food intake by a subject. In some embodiments, the composition is more effective or potent at treating a metabolic disorder when administered before food intake. For example, the composition may be administered about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject.

In some embodiments, the composition is administered after food intake by the subject. In some embodiments, the composition is more effective or potent at treating a metabolic disorder when administered after food intake. For example, the composition may be administered at least about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject. For example, the formulation may be administered at most about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject.

Compositions provided herein include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

In some embodiments, the composition may be a therapeutic composition, a pharmaceutical composition, or a dietary supplement composition.

A therapeutic composition, as disclosed herein, may include carriers and excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically acceptable excipients suitable for use in the disclosure include granulating agents, binding agents, lubricating agents, disintegrating agents, sweetening agents, glidants, anti-adherents, anti-static agents, surfactants, antioxidants, gums, coating agents, coloring agents, flavoring agents, dispersion enhancer, disintegrant, coating agents, plasticizers, preservatives, suspending agents, emulsifying agents, plant cellulosic material and spherization agents, and any combination thereof.

Non-limiting examples of pharmaceutically acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A therapeutic composition may be substantially free of preservatives. In some applications, the composition may contain at least one preservative.

A therapeutic composition may be encapsulated within a suitable vehicle, for example, a liposome, a microsphere, or a microparticle. Microspheres formed of polymers or proteins may be tailored for passage through the gastrointestinal tract directly into the blood stream. Alternatively, the compound may be incorporated and the microspheres, or composite of microspheres, and may be implanted for slow release over a period of time ranging from days to months.

A therapeutic composition may be formulated as a sterile solution or suspension. The therapeutic compositions may be sterilized by conventional techniques or may be sterile filtered. The resulting aqueous solutions may be packaged for use as is or lyophilized. The lyophilized preparation of the microbial composition may be packaged in a suitable form for oral administration, for example, capsule or pill. In one embodiment, the capsule may comprise of enteric polymers that do not disintegrate until after the capsule exits the stomach of the subject.

In one embodiment, the capsule ingredient may comprise of methyl cellulose, hydroxypropyl methylcellulose phthalate, propylene glycol and derivatives of such polymers.

The compositions may be administered topically and may be formulated into a variety of topically administrable compositions, such as solutions, suspensions, lotions, gels, pastes, medicated sticks, balms, creams, and ointments. Such pharmaceutical compositions may contain solubilizers, stabilizers, tonicity enhancing agents, buffers and preservatives.

The compositions may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, may be used.

In practicing the methods of treatment or use provided herein, therapeutically effective amounts of the microbial compositions described herein are administered in pharmaceutical compositions to a subject having a disease or condition to be treated. In some embodiments, the subject is a mammal such as a human. A therapeutically effective amount may vary widely depending on the severity of the disease, the age and relative health of the subject, potency of the formulation, and other factors. Subjects may be, for example, humans, elderly adults, adults, adolescents, pre-adolescents, children, toddlers, infants, or neonates.

A subject may be a patient. A subject may be an individual enrolled in a clinical study. A subject may be a laboratory animal, for example, a mammal. A subject may be a human.

Pharmaceutical compositions may be formulated using one or more physiologically acceptable carriers comprising excipients and auxiliaries, which facilitate processing of the microorganisms into preparations that may be used pharmaceutically. Formulation may be modified depending upon the route of administration chosen. Pharmaceutical compositions described herein may be manufactured in a conventional manner, for example, by means of conventional mixing, dissolving, granulating, vitrification, spray-drying, lyophilizing, levigating, encapsulating, entrapping, emulsifying or compression processes.

In some embodiments, the pharmaceutical composition is manufactured in a dry form, for example, by spray-drying or lyophilization. In some embodiments, the formulation is prepared as a liquid capsule to maintain the liquid form of the microbes.

Compositions provided herein may be stored at any suitable temperature. The formulation may be stored in cold storage, for example, at a temperature of about −80° C., about −20° C., about −4° C., or about 4° C. The storage temperature may be, for example, about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C. In some embodiments, the storage temperature is between about 2° c. to about 8° c. storage of microbial compositions at low temperatures, for example from about 2° C. to about 8° C., may keep the microbes alive and increase the efficiency of the composition, for example, when present in a liquid or gel formulation. Storage at freezing temperature, below 0° C., with a cryoprotectant may further extend stability.

The pH of the composition may range from about 3 to about 12. The pH of the composition may be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units.

The pH of the composition may be, for example, about 3, about 4, about 5, about 6, about 7, about 8, about 9, about 10, about 11, or about 12 pH units. The pH of the composition may be, for example, at least 3, at least 4, at least 5, at least 6, at least 7, at least 8, at least 9, at least 10, at least 11 or at least 12 pH units. The pH of the composition may be, for example, at most 3, at most 4, at most 5, at most 6, at most 7, at most 8, at most 9, at most 10, at most 11, or at most 12 pH units. If the pH is outside the range desired by the formulator, the pH may be adjusted by using sufficient pharmaceutically acceptable acids and bases. In some embodiments, the pH of the composition is between about 4 and about 6.

Pharmaceutical compositions containing microbes described herein may be administered for prophylactic and/or therapeutic treatments. In therapeutic applications, the compositions may be administered to a subject already suffering from a disease or condition, in an amount sufficient to cure or at least partially arrest the symptoms of the disease or condition, or to cure, heal, improve, or ameliorate the condition. Microbial compositions may also be administered to lessen a likelihood of developing, contracting, or worsening a condition. Amounts effective for this use may vary based on the severity and course of the disease or condition, previous therapy, the subject's health status, weight, and response to the drugs, and the judgment of the treating physician.

Multiple therapeutic agents may be administered in any order or simultaneously. If simultaneously, the multiple therapeutic agents may be provided in a single, unified form, or in multiple forms, for example, as multiple separate pills. The composition may be packed together or separately, in a single package or in a plurality of packages. One or all of the therapeutic agents may be given in multiple doses. If not simultaneous, the timing between the multiple doses may vary as much as about a month.

Compositions described herein may be administered before, during, or after the occurrence of a disease or condition, and the timing of administering the composition may vary. For example, the microbial composition may be used as a prophylactic and may be administered continuously to subjects with a propensity to conditions or diseases in order to lessen the likelihood of the occurrence of the disease or condition. The microbial compositions may be administered to a subject during or as soon as possible after the onset of the symptoms. The administration of the microbial compositions may be initiated within the first 48 hours of the onset of the symptoms, within the first 24 hours of the onset of the symptoms, within the first 6 hours of the onset of the symptoms, or within 3 hours of the onset of the symptoms. The initial administration may be via any route practical, such as by any route described herein using any formulation described herein. A microbial composition may be administered as soon as is practicable after the onset of a disease or condition is detected or suspected, and for a length of time necessary for the treatment of the disease, such as, for example, from about 1 month to about 3 months. The length of treatment may vary for each subject.

In an aspect, compositions may be administered in combination with another therapy, for example, immunotherapy, chemotherapy, radiotherapy, anti-inflammatory agents, anti-viral agents, anti-microbial agents, and anti-fungal agents.

In another aspect, compositions may be packaged as a kit. In some embodiments, a kit includes written instructions on the administration/use of the composition. The written material may be, for example, a label. The written material may suggest conditions methods of administration. The instructions provide the subject and the supervising physician with the best guidance for achieving the optimal clinical outcome from the administration of the therapy. The written material may be a label. In some embodiments, the label may be approved by a regulatory agency, for example the U.S. Food and Drug Administration (FDA), the European Medicines Agency (EMA), or other regulatory agencies.

### G. METHODS OF TREATMENT

The disclosure provides methods of treatment of a subject. The subject may be suffering from or may be predisposed to symptoms related to disorders that originate from a disbalance of gut microbiome. Altering the composition of a microbiome in such a subject may have desired health consequences. Compositions of the present disclosure may be administered as a therapeutic and/or a dietary supplement for treating a health condition. Treatments designed to alter the host microbiome(s) may result in a reduction of patient symptoms, prevention of disease, and or treatment of the disease or health condition. For example, modification of the gut microbiome may reduce the risk for health conditions such as metabolic disorders, neurological or behavioral disorders.

Therefore, the present disclosure provides methods for the restoration of a microbial habitat of a subject to a healthy state. The method may comprise microbiome correction and/or adjustment including for example, replenishing native microbes, removing pathogenic microbes, administering prebiotics, and growth factors necessary for microbiome survival. In some embodiments, the method also comprises administering antimicrobial agents such as antibiotics.

Based on the microbiome profile, the present disclosure provides methods for generalized-treatment recommendation for a subject as well as methods for subject-specific treatment recommendation. Methods for treatments may comprise one of the following steps: determining a first ratio of a level of a subject-specific microbiome profile to a level of a second microbiome profile in a biological sample obtained from at least one subject, detecting a presence or absence of a disease in the subject based upon the determining, and recommending to the subject at least one generalized or subject-specific treatment to ameliorate disease symptoms.

The diseases that may be treated include those in which the alteration of the gut microbiome is beneficial to a subject suffering from the disease. Specifically, the gut microbiome of a subject may be scarce on the population of at least one live, active gut bacteria selected from the group consisting of: Akkermansia muciniphila, Clostridium butyricum, Clostridium beijerinckii, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Eubacterium hallii, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combinations thereof.

In one embodiment, the gut microbe that are in lower levels, in a subject suffering from or predisposed to disorders as mentioned above, is Akkermansia muciniphila. In another embodiment, the gut microbe that are in lower levels, in a subject suffering from or predisposed to disorders as mentioned above, is Eubacterium hallii. In another embodiment, the gut microbe that are in lower levels, in a subject suffering from or predisposed to disorders as mentioned above belongs to the genus Clostridia, Eubacteria, Bifidobacteria, Anaerostipes, Coprococcus, Bacteroides, Blautia, Ruminococcus, Faecalibacterium, Oscillospira, Streptococcus, and Roseburia.

In another embodiment, the subject has more than one gut microbe in levels lower than their levels in a normal subject. Specifically, the microbes may be at least one or more of: Akkermansia muciniphila, Clostridium butyricum, Clostridium beijerinckii, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Eubacterium hallii, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combinations thereof.

Measuring the microbiome of subjects may show that microbiomes lacking various strains of microorganisms result in a health condition and/or disease state. Restoring one or more lacking strains (e.g., via a bacterial strain such as A. muciniphila) results in alteration of the health condition. Some non-limiting examples include altering the gut microbiome such that the host has an increased capacity for energy harvest, increased insulin sensitivity, and/or decreased appetite, decreased anxiety, decreased hyperalgesia, decreased behavioral issues like anxiety, regularized bowel movements, decreased bloating, decreased constipation.

Some non-limiting heath conditions that can be affected by an imbalance of microbial population include, for example, Type 2 Diabetes Mellitus (T2DM), preterm labor, chronic fatigue syndrome, skin conditions such as acne, allergies, autism, asthma, depression, hypertension, metabolic syndrome, obesity, lactose intolerance, oral thrush, ulcerative colitis, drug metabolism, vaginosis, atopic dermititus, psoriasis, Type I Diabetes Mellitus (T1DM), Multiple Sclerosis, neurological disorders such as Parkinson's disease, Clostridium Difficile infection, heart disease, diabetic foot ulcers, bacteremia, infantile colic, cancer, cystic fibrosis, multiple sclerosis, urinary tract infection, radiation enteropathy, drug metabolism, dental cavities, and halitosis.

Measuring the microbiome of subjects may be conducted by analysing body fluid samples or conducting microbial analyses on faeces of the subject. Candidate strains can be found in scientific literature and studies. These candidate strains may be used as parameters to be measured from subject's microbiome analyses. By comparing ideal levels of each microbe in a healthy individual to the levels of that microbe in the subject, a conclusion can be made regarding the levels of that microbe in the gut of the subject. Accordingly, the compositions as disclosed herein, may be administered to replenish and reinstate the population of the deficient gut microbe as identified by the microbial analyses.

### I. Dosing

The appropriate quantity of a therapeutic composition to be administered, the number of treatments, and unit dose may vary according to a subject and/or the disease state of the subject.

Pharmaceutical compositions described herein may be in unit dosage forms suitable for single administration of precise dosages. In unit dosage form, the formulation may be divided into unit doses containing appropriate quantities of one or more microbial compositions. The unit dosage may be in the form of a package containing discrete quantities of the formulation. Non-limiting examples are liquids in vials or ampoules. Aqueous suspension compositions may be packaged in single-dose non-resealable containers. The composition may be in a multi-dose format. Multiple dose resealable containers may be used, for example, in combination with a preservative. Formulations for parenteral injection may be presented in unit dosage form, for example, in ampoules, or in multi-dose containers with a preservative.

The dosage may be in the form of a solid, semi-solid, or liquid composition. Non-limiting examples of dosage forms include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

A microbe may be present in any suitable concentration in a pharmaceutical composition. The concentration of a microbe may be for example, from about 10 \hat{ }1 to about 10\hat{ }18 CFU or AFU per gram of the media. The concentration of a microbe may be, for example, at least 10\hat{ }1, at least 10\hat{ }2, at least 10\hat{ }3, at least 10\hat{ }4, at least 10\hat{ }5, at least 10\hat{ }6, at least 10\hat{ }7, at least 10\hat{ }8, at least 10\hat{ }9, at least 10\hat{ }10, at least 10\hat{ }11, at least 10\hat{ }12, at least 10\hat{ }13, at least 10\hat{ }14, at least 10\hat{ }15, at least 10\hat{ }16, at least 10\hat{ }17, or at least 10\hat{ }18 CFU or AFU per gram. The concentration of a microbe may be, for example, at most 10\hat{ }1, at most 10\hat{ }2, at most 10\hat{ }3, at most 10\hat{ }4, at most 10\hat{ }5, at most 10\hat{ }6, at most 10\hat{ }7, at most 10\hat{ }8, at most 10\hat{ }9, at most 10\hat{ }10, at most 10\hat{ }11, at most 10\hat{ }12, at most 10\hat{ }13, at most 10\hat{ }14, at most 10\hat{ }15, at most 10\hat{ }16, at most 10\hat{ }17, or at most 10\hat{ }18 CFU or AFU per gram. In some embodiments, the concentration of a microbe is from about 10\hat{ }8 CFU to about 10\hat{ }9 CFU or AFU per gram. In some embodiments, the concentration of a microbe is about 10\hat{ }8 CFU or AFU per gram. In some embodiments, the concentration of a microbe is about 10\hat{ }9 CFU or AFU per gram.

Pharmaceutical compositions, as presented here, may be formulated with any suitable therapeutically effective concentration of prebiotic. For example, the therapeutically effective concentration of a prebiotic may be at least about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically effective concentration of a prebiotic may be at most about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically effective concentration of a prebiotic may be about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. In some embodiments, the concentration of a prebiotic in a pharmaceutical composition is about 70 mg/ml. In some embodiments, the prebiotic is inulin.

Pharmaceutical compositions, as in the present disclosure, may be administered for example, 1, 2, 3, 4, 5, or more times daily. In an embodiment, the pharmaceutical compositions may be administered, for example, daily, every other day, three times a week, twice a week, once a week, or at other appropriate intervals for treatment of the condition.

While the foregoing invention has been described in some detail for purposes of clarity and understanding, it will be clear to one skilled in the art from a reading of this disclosure that various changes in form and detail may be made without departing from the true scope of the invention. For example, all the compositions and methods described above may be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### H. EXAMPLES

### Example 1: Improved Growth Phase of Amuc-i09 v. Amuc-OG

Objective: The purpose of the study was to determine growth differences between AMUC i09 and AMUC OG.

Methods: AMUC colonies were grown on VEG agar plates for 5 days until single colonies were identified. Single colonies were inoculated into two replicate 8 ml tubes containing VEG media at 37C shaking at 180 rpm. Optical density at 600 nm (OD600) was measured at different time intervals. As shown in FIG. 3, Amuc-i09 is characterized by a faster doubling time as compared to Amuc-OG at exponential growth. There was no difference in pH and glucose consumption between the two strains during the exponential growth phase.

### Example 2: Improved Pelleting Properties of Amuc-i09

Objective: The purpose of this experiment was to identify physiological differences between Amuc-i09 and Amuc-OG.

Methods: Amuc-i09 and Amuc-OG colonies were grown on VEG agar plates for 5 days until single colonies were identified. Single colonies were inoculated into two replicate 8 ml tubes containing VEG-media at 37C shaking at 180 rpm. Optical density at 600 nm (OD600) was measured at different time intervals throughout the growth phase in 8 ml tubes. Once OD600 reached 1.0, the 8 ml culture was used to inoculate 100 ml bottles. The 100 ml bottles were grown to OD600 of 1.0 and was subsequently subcultured into 500 ml bottles. The 500 ml bottles were grown to OD600 of 1.0 and was centrifuged at 7,500 rpm for 20 minutes. Amuc-i09 cells displayed improved pelleting properties as compared to Amuc-OG. This resulted in a difference in dry weight, as shown in Table 2, when the product is lyophilized.

TABLE 2
Difference in dry weight of pellets

| | | Dry | Total | |
|---|---|---|---|---|
| Strain | Cryoprotectant | Weight (g) | CFU | CFU/g |
| Amuc-i09 | 10% Sucrose | 4 | 8.72E+11 | 2.18E+11 |
| Amuc-OG | 10% Sucrose | 21.3 | 8.29E+11 | 3.89E+10 |

### Example 3: Treatment of a Metabolic Disorder with a Composition Comprising Amuc-i09

Objective: To treat a metabolic disorder in a human with a composition comprising Amuc-i09.

Methods: An individual suffering from a metabolic disorder is treated with a composition comprising AMuc-i09. The composition comprises Amuc-i09 at a range of about 10\hat{ }9 CFU to 10\hat{ }10 CFU and inulin.

The expected delivery form of the oral composition is an enteric-coated (e.g., pH sensitive polymer Eudragit FS30D) pill that can protect against stomach acidity and deliver to the ileum/upper colon region of the subject. The enteric coating is designed to dissolve at a pH greater than about 6.5-7. In some embodiments, the oral composition is administered as a liquid capsule. The subject is administered the composition before food intake (e.g., 1 hour before meals) twice daily for fourteen consecutive days. In some cases, the composition is administered simultaneously with food intake.

The microbial composition alters the microbial habitat of the gut of the subject to that of a healthy subject. The subject loses weight. The subject's metabolic condition, for example, obesity, insulin insensitivity, T2DM, and/or T1DM is treated by the composition.

### Example 4: Sequencing Amuc-i09

Objective: To sequence Amuc-i09 and identify genomically unique sequences

Methods: Amuc-i09 was sequenced using PacBio RSII and was assembled into one contig which seemed to be the full chromosome, indicated through repetitive, overlapping ends.

After assembly, a circular contig comprising the full genome was obtained. We built a database of all publicly available Akkermansia muciniphila proteins by downloading all the proteins from ref seq assigned to Akkermansia muciniphila. A reciprocal blast search of predicted Amuc-i09 protein sequences against this database combined with the genome of AMUC-OG revealed a total of 2 unique genes (diamond blastp with max e-value of 1e-03), SEQ ID NO: 1 and SEQ ID NO: 2. Out of these genes, 0 has a hit to genes of organisms outside of the Akkermansia clade (blastp against refseq).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. (canceled)

2. (canceled)

3. (canceled)

4. (canceled)

5. A composition comprising bacteria, wherein the bacteria comprise one mor more of:
an Akkermansia muciniphila strain as deposited at the American Type Culture Collection (ATCC) under ATCC Accession No. PTA-126838;
an Akkermansia muciniphila strain comprising one or more nucleic acids encoding a polypeptide comprising the amino acid sequence set forth in SEQ ID NO:1, a polypeptide comprising the amino acid sequence set forth in SEQ ID NO:2, or both;
an Akkermansia muciniphila strain comprising a polypeptide comprising the amino acid sequence set forth in SEQ ID NO: 1 and a polypeptide comprising the amino acid sequence set forth in SEQ ID NO: 2; and
bacteria that express a polypeptide comprising the amino acid sequence set forth in SEQ ID NO:1, a polypeptide comprising the amino acid sequence set forth in SEQ ID NO:2, or both.

6. (canceled)

7. The composition of claim 5, further comprising one or more additional microbes having a 16S rRNA sequence comprising at least 97% identity to the full length of a 16S rRNA sequence of a microbe selected from the group consisting of: Anaerostipes caccae, Anaerobutyricum hallii, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcusfaecis, and any combinations thereof.

8. (canceled)

9. The composition of claim 7, wherein the composition comprises at least 10\hat{ }5 AFUs/g of each of the one or more additional microbes.

10. The composition claim 5, wherein
the composition further comprises at least one preservative;
the composition further comprises an enteric coating; and/or
the composition further comprises lyophilized bacteria.

11. (canceled)

12. The composition claim 5, wherein the composition further comprises a prebiotic.

13. The composition of claim 12, wherein the prebiotic is selected from the group consisting of inulin, green banana, reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, b-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof.

14. The composition of claim 12, wherein the prebiotic is inulin.

15. (canceled)

16. (canceled)

17. The composition of claim 5, wherein the bacteria are viable or wherein the bacteria are non-viable.

18. (canceled)

19. The composition of any one of claims 5-18, wherein the composition is formulated as a
pharmaceutical formulation;
nutritional supplement;
dietary supplement and/or;
medical food.

20. (canceled)

21. (canceled)

22. (canceled)

23. The composition of claim 5, wherein the composition is dairy-free.

24. The composition of claim 5, wherein the composition comprises substantially no animal products.

25. The composition of claim 5, wherein the composition is in the form of a pill, a capsule, a lozenge, a food bar, or a gummy ball.

26. A method comprising administering the composition of claim 5 to a subject in need thereof.

27. The method according to claim 26, wherein the composition is administered in
an amount effective to treat a metabolic disorder in the subject.

28. The method of claim 27, wherein the subject is administered at least one dose daily of the composition.

29. The method of claim 27, wherein the subject is administered at least 1×10\hat{ }8 CFU of the Akkermansia muciniphila strain in a single dose.

30. The method of claim 27, wherein the metabolic disorder is selected from the group consisting of or consisting essentially of: insulin-resistance based disorders, gut-related disorders, insulin-sensitivity based disorders, type-1-diabetes, type-2-diabetes, and obesity.

31. A method comprising culturing the composition of claim 5 under anaerobic conditions in/on a growth medium.

32. The method of claim 31, wherein the growth medium is a vegetable-based medium.

33. The method of claim 32, wherein the vegetable-based medium is VEG-medium.
